(19) United States
(12) Patent Application Publication
(10) Pub. No.: US 2009/0018462 A1
(43) Pub. Date: Jan. 15, 2009

(54) Neurologic System and Associated Methods

(76) Inventor: John Bell, Sandy, UT (US)

Correspondence Address: THORPE NORTH & WESTERN, LLP., P.O. Box 1219, SANDY, UT, 84091-1219, US

(21) Appl. No.: 12/091,274

(22) PCT Filed: Oct. 24, 2006
(86) PCT No.: PCT/US06/41826

§ 371 Date: Oct. 2, 2008

Related U.S. Application Data
(60) Provisional application No. 60/730,122, filed on Oct. 24, 2005.

(51) Int. Cl. A61B 5/04

(52) U.S. Cl. 600/544

(57) ABSTRACT

The present invention includes systems and methods for assessing stimulatory effects on a neurologic system. One method may include steps of stimulating the neurologic system, monitoring at least one neurologic state for effects of the stimulation to the neurologic system, gathering multi-dimensional data from the monitoring of the at least one neurological state, and analyzing the multi-dimensional data to determine multi-dimensional interactions between the stimulation and the effects on the at least one neurological state. Various neurologic states are considered to be within the scope of the present invention, including, without limitation, hypnotic, analgesia, relaxation, stress, depression, anxiety, allostasis, immune response, and combinations thereof.

### PRIORITY DATA

This application claims the benefit of PCT Application No. PCT/US06/41826, filed Oct. 24, 2006, which claims the benefit of U.S. Provisional Patent Application Ser. No. 60/730,122, filed on Oct. 24, 2005, both of which are incorporated herein by reference in their entirety.

### FIELD OF THE INVENTION

The present invention provides systems and methods related to neurologic research and treatment. Accordingly, the present invention involves the fields of neuroscience, biology, and medicine.

### BACKGROUND OF THE INVENTION

The field of neuroscience has become a rapidly growing area of clinical medicine and scientific research in humans, animals, and even insects. Neuroscience researchers attempt the daunting task of manipulating, characterizing, and understanding extremely complex neural interactions, from the cellular level to neural networks. Though experimental research encompasses basic neuronal function, intracellular mechanisms, small inter-neuronal interactions, neuronal network function, and complex behavioral analysis, in some cases the basic research methodology may be very similar.

Though experimental neuroscience research encompasses basic neuronal function, intracellular mechanisms, small inter-neuronal interactions, neuronal network function, and complex behavioral analysis, the same classic linear research methodology tends to be used for all of these areas. In such cases, a researcher may identify a neurological question or a neurologically related issue and proceed methodically in a linear path in an attempt understand such a question or issue. Such an approach may include affecting the neurologic system in some meaningful manner, observing the response of the affect, and analyzing large quantities of repetitious data in an attempt to eventually observe some meaningful pattern. For example, studying the effects of a new drug on several neurologic states may entail a research paradigm that tests the drug for its effects on one neurologic state and performs a statistical analysis of the results, then repeat the experimental process again for the second neurologic state, then repeats the process for the third state, etc. Such an approach to research limits data collection to a two dimensional linear progression of events. Such a linear process is inefficient and time consuming.

Additionally, many previous methods of detecting or measuring various neuronal responses and associated neurologic states, especially in clinical medicine, have relied upon relatively subjective means of monitoring gross changes in physiological measures such as physical movement, pulse, respiration, or subjective analogs like the Visual Analog Scale for pain measurement. The ability to induce or control neurologic states is has been limited almost exclusively to pharmaceutical means. Without selective and objective metrics, the ability to manipulate or maintain specific neurologic states with precision and accuracy, by pharmaceutical and other means, remains very limited. Additionally, research to establish that specific neurologic states result from given pharmacological or non-pharmacological stimulation variables, often requires data collected from dozens to hundreds of controlled experiments to achieve acceptable levels of statistical significance.

It would thus be beneficial to clinical medicine and neuroscience research to develop a neurologic system employing objective and multi-dimensional neurologic monitoring with multi-dimensional data processing and analysis capabilities and means to stimulate specific neurologic states.

### SUMMARY OF THE INVENTION

Accordingly, the present invention provides monitoring methods for sensing biological and other responses reflecting various aspects of the nervous system associated with specific neurologic states. The invention also provides a multi-dimensional data processing method for analyzing neurologic sensor data to discriminate, identify, and characterize one or more neurologic states of a complex neurologic system. Additionally, the present invention also provides controlled stimulation methods to induce, manipulate, or maintain one or more neurologic states.

Aspects of the present invention combine elements of several different technologies in combinations that create new system configurations, methods, and uses that have not previously been described by any single patent. Skills in several core technologies are required for the development and practice of the invention, these technology fields include therapeutic medical science, neurologic monitoring, data processing and analysis, and control system engineering. Therapeutic medical science skills include the fields of pharmacology, anesthesia, psychology, immunology, etc. Neurologic monitoring technology skills may includes the field of neuroscience and one of more skill in fields such as electroencephalography, electrocardiography, electromyography, biochemical assay, MRI, psychological assessment, etc. Data processing and analysis skills may include the fields of software and firmware development, database design, data mining methods, medical expert systems and medical informatics. Control system engineering is a required skill area for the development and practice of a feedback means for the control of the neurologic stimulation element that is used on some configurations of the invention. The technical aspects of the invention and its several configurations are easily understood by those skilled in each of these fields. However, due to the inherent diversity of technology employed in the invention, all technical aspects of the invention would not be obvious to those skilled in the art of only one aspect of the invention.

The invention brings together component technology elements in several configurations to create new neuroscience tools and capabilities that have not previously been described to satisfy unmet needs for neuroscience medicine and research, particularly the need for objective and precise neurologic state monitoring and for a means to precisely stimulate and manipulate neurologic states.

In one aspect, a method of assessing stimulatory effects on a neurologic system. Such a method may include steps of stimulating the neurologic system, monitoring at least one neurologic state for effects of the stimulation to the neurologic system, gathering multi-dimensional data from the monitoring of the at least one neurological state, and analyzing the multi-dimensional data to determine relationships between the stimulation and the effects on the at least one neurological state. Various neurologic states are considered to be within the scope of the present invention, including, without limitation, hypnotic, analgesia, relaxation, stress, depression, anxiety, allostasis, immune responses, and combinations thereof. Additionally, analyzing the multi-dimensional data can occur over short time intervals. In one aspect, for example, the step of analyzing the multi-dimensional data may occur in less than 3 minutes. In another aspect, the step of analyzing the multi-dimensional data may occur in less than 1 minute. In yet another aspect, the step of analyzing the multi-dimensional data may occur in less than 30 seconds.

In another aspect of the present invention, a method of assessing stimulatory effects on a neurologic system is provided. The method may include steps of stimulating the neurologic system, monitoring with multiple monitors at least one neurologic state for effects of said stimulation to the neurologic system, gathering data from the multiple monitors of the at least one neurological state, and analyzing the data to determine changes in the neurological state due to the stimulation. Additionally, the method may further include a step of varying the stimulation of the neurologic system as a result of changes in the neurological state.

In yet another aspect, a system for assessing stimulatory effects on a neurologic system of a subject is provided. The system may include a neurological stimulator configured to be functionally coupled to the subject, multiple neurological monitoring elements configured to be functionally coupled to the subject in order to monitor at least one neurologic state, and a neurologic data processing element configured to analyze multi- dimensional data from the at least one neurologic state. In one aspect, the multiple neurological monitoring elements are configured to physically contact a skin surface of the subject. In another aspect, the multiple neurological monitoring elements are configured to not physically contact a skin surface of the subject. Additionally, the neurologic data processing element can rapidly analyze multi-dimensional data. In one aspect, for example, the neurologic data processing element is capable of analyzing the multi-dimensional data in less than 3 minutes. In another aspect, the neurologic data processing element is capable of analyzing the multi-dimensional data in less than 1 minute. In yet another aspect, the neurologic data processing element is capable of analyzing the multi-dimensional data in less than 30 seconds.

In a further aspect, a method of monitoring a neurologic state of a neurologic system is provided. In one aspect, the method may include steps of monitoring at least one neurologic state of the neurologic system, gathering multi-dimensional data from the monitoring of the at least one neurological state, and analyzing the multi-dimensional data to evaluate the at least one neurological state.

### BRIEF DESCRIPTION OF THE DRAWINGS

FIG. 1 is a schematic view of a neurologic system in accordance with an aspect of the present invention.

FIG. 2 is a schematic view of a neurologic system in accordance with another aspect of the present invention.

FIG. 3 is a schematic view of a neurologic system in accordance with yet another aspect of the present invention.

FIG. 4 is a schematic view of a neurologic system in accordance with a further aspect of the present invention.

FIG. 5 is a graphical view of a simulated data in accordance with an aspect of the present invention.

FIG. 6 is a graphical view of a simulated data in accordance with an aspect of the present invention.

FIG. 7 is a graphical view of a simulated data in accordance with an aspect of the present invention.

FIG. 8 is a graphical view of a simulated data in accordance with an aspect of the present invention.

FIG. 9 is a graphical view of a simulated data in accordance with an aspect of the present invention.

FIG. 10 is a graphical view of a data in accordance with an aspect of the present invention.

### DETAILED DESCRIPTION OF THE INVENTION

Before the present systems and methods relating to neurologic research and treatment are disclosed and described, it is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and, “the” include plural referents unless the context clearly dictates otherwise. Thus, for example, reference to “a neurologic state” includes reference to one or more of such states.

### Definitions

In describing and claiming the present invention, the following terminology will be used in accordance with the definitions set forth below.

As used herein, “Relational Data Characterization” (RDC), may include any analysis technique used to find and/or depict the nature of relationships that exist within multi-dimensional data sets. RDC may further facilitate the ability to view or describe functional relationships in multi-dimensional sets of discrete or signal data. In some aspects, RDC may include any method of data processing that can concurrently processes multi-dimensional data sets to characterize one or more relationships that may occur within the multi-dimensional data sets as input variables change.

As used herein, “Multi-dimensional data” may include data that reflects more than one aspect/characteristic of a neurologic state. As an example, an ohmmeter is an instrument that only provides one measured characteristic, or “discrete data element,” which is “electrical resistance”. Conversely, an oscilloscope is a single instrument that can measure complex multidimensional aspects of a signal, or contiguous dependent data, namely amplitude, frequency, and modulation for example.

As used herein, “discrete data elements” refers to data values that are disjunctive representations of events, conditions, responses, etc. In other words, each data element is independent of the immediately preceding and following data elements. Non-limiting examples of discrete data elements may include (1) tables of gene expressions and (2) periodic average values of instrument readings such as periodic blood pressure values. As such, a “discrete monitor” is an instrument that monitors periodic discrete data elements.

As used herein, the terms “contiguous data,” “dependent data,” and “contiguous dependent data” may be used interchangeably, and refer to data where each data element is dependent upon its immediately preceding data element. One example may include signal data, a continuously varying data stream depicting the waveforms characterized by sensor outputs, such as the electroencephalogram (EEG) or electrocardiogram (ECG), from which multidimensional data may be derived. The signal waveform output by an arterial blood pressure monitor is an example of “contiguous data” or “dependent data,” but a collection of periodic systolic and diastolic blood pressure values would be “discrete data elements.” As such, a “continuous data monitor” or a “signal monitor” is an instrument that monitors a contiguous, or dependent, data stream.

As used herein, “noninvasive” refers to a form of stimulation that does not require a rupture or puncture a biological membrane or structure with a mechanical means across which an electrode or other stimulatory means is passed. Surface electrodes are one example of a noninvasive stimulatory means that is well recognized in the neurological arts. “Invasive” refers to a form of stimulation that requires a rupture or puncture a biological membrane or structure with a mechanical means across which an electrode or other stimulatory or sensory means is passed. Implantable electrodes are one example of an invasive stimulation or sensory means.

As used herein, “functionally coupled” refers to any form of interconnection between components that may be either physical or non-physical. Examples of physical connections include electrical wire, surface or percutaneous electrodes, fiber optic cable, etc. Non-physical connections may, include without limit, optical coupling, magnetic coupling, wireless communications, displacement current sensing, quantum data teleportation, etc.

As used herein, “sensor” or “neurologic sensor” refers to any means of actively or passively collecting information or data about neurologic states or neurologic responses. This may include, without limitation, any form of biological sensor such as EEG, ECG, biological assays, etc., observational monitoring such as psychological observations, or any other instruments to collect information, including assessment surveys and questionnaires, that may indicate neurologic states or conditions.

As used herein, “subject” refers to an animal or insect that possesses at least a rudimentary nervous system. Examples of subjects include humans, and may also include other animals such as horses, pigs, cattle, dogs, cats, rabbits, rats, birds, anurans, reptiles, aquatic mammals, fish, etc.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof. As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### The Invention

The present invention provides the application of unique combinations of technologies that can depict, analyze, and/or affect neurologic states to provide important new capabilities in the fields of clinical medicine and neuroscience research. These capabilities may include 1) multidimensionality—the ability to process and analyze complex data from one or multiple neurologic sensors or other sources; 2) discrimination—the ability to specifically identify and depict one or multiple neurologic states; 3) concurrency—the ability to process multi-dimensional neurologic sensor data concurrently, or within a very brief period of time; 4) characterization—the ability to process multi-dimensional neurologic sensor data to quantify or otherwise depict characteristics of specific neurologic states; 5) relationality—the ability to process multi-dimensional neurologic sensor data to identify and depict relationships that reflect specific neurologic states or changes in states; 6) stimulation—the ability to affect changes in neurologic states by various forms of neurologic stimulation; and 7) control—the ability to use information from multi-dimensional neurologic data analysis as feedback to adjust neurologic stimulation parameters to manipulate or maintain one or more specific neurologic states.

As such, the present invention provides methods and systems for evaluating neurological states. Such evaluation may include diagnosing and treating various neurological conditions in addition to monitoring a neural state of a subject. Additionally, systems and methods according to aspects of the present invention may prove to be valuable tools in performing neurologic research. Such research may be performed in a clinical or non-clinical environment on a subject. It should be noted, however, that the scope of the present invention is not limited to specific areas of research or medicine, but may be applicable to any application relating to the monitoring, diagnosis, treatment, and/or neural study of humans and animals.

Systems and methods according to aspects of the present invention are provided that facilitate and enhance neurological evaluation by detecting and measuring one or more neurologic states produced in a subject by pharmacological and/or non-pharmacological stimulation mechanisms. It has been discovered that neurological data processing methods, including Relational Data Characterization (RDC) techniques, can be used to concurrently process data from multiple neurologic state sensors to identify and depict changes that occur in specific neurologic states associated with changes in various neurologic stimulation parameters.

Neurologic evaluation of a subject for medical and research purposes would be greatly enhanced by a system in which a large sample of potentially relevant input and output data could be concurrently collected and processed to depict how a wide range of output variables actually change in response to changes to the input parameters. This “shotgun” approach provides the base of relevant data needed to efficiently develop useful testable theories, and it identifies the important parameters that have an effect on specific categories of results. In simple terms, a research trial with such a system describes how, as opposed to simply demonstrating whether on not, a plurality of output results are functionally related to changes in the values of a plurality of input parameters. Such an approach allows the collection of many neurologic state evaluations in single trials. Results of concurrent neurologic monitoring may create proportionately larger n-dimensional neurologic evaluation data sets.

As such, the innovative system described herein was developed to facilitate the discovery process in neurologic state research and to improve neurologic evaluation and therapy in medical settings. Examples of such neurologic states might include hypnotic, analgesia, relaxation, stress, depression, anxiety, allostasis, immune responses, or any other state in which changes in neural or physiologic processes can be detected or measured. This system of conducting neurologic state evaluations may allow medical professionals and investigators to quickly collect a relatively large body of data and then identify a range of potentially relevant associations that may exist between input stimuli and changes in a number of specific neurologic states. Stimuli with little or no effect on relevant neurologic states can be excluded from further evaluation if desired. By providing a concurrent view of such a large multi-dimensional data set, rather than individual data points, and providing a means to depict relevant associations that occur between input parameters and a range of output results, this system of neurologic evaluation gives investigators a much better perspective and understanding of the processes that occur in their research experiments. This approach is expected to greatly accelerate the scientific discovery process and the development of useful and accurate theories to advance neurologic science, as well as providing more effective methods for neurologic evaluation for medical purposes.

As has been described, numerous system configurations for neurologic evaluation are contemplated that encompass both clinical and non-clinical applications are now generally described. Specific details regarding individual elements are discussed below. Though various systems are described herein, it should be understood that these example embodiments are merely exemplary, and no limitation should be implied by their organization or the names applied to each configuration. Accordingly, the following embodiments are merely descriptive examples of possible collections of the various elements of the present invention that may be useful in particular neurologic evaluation tasks.

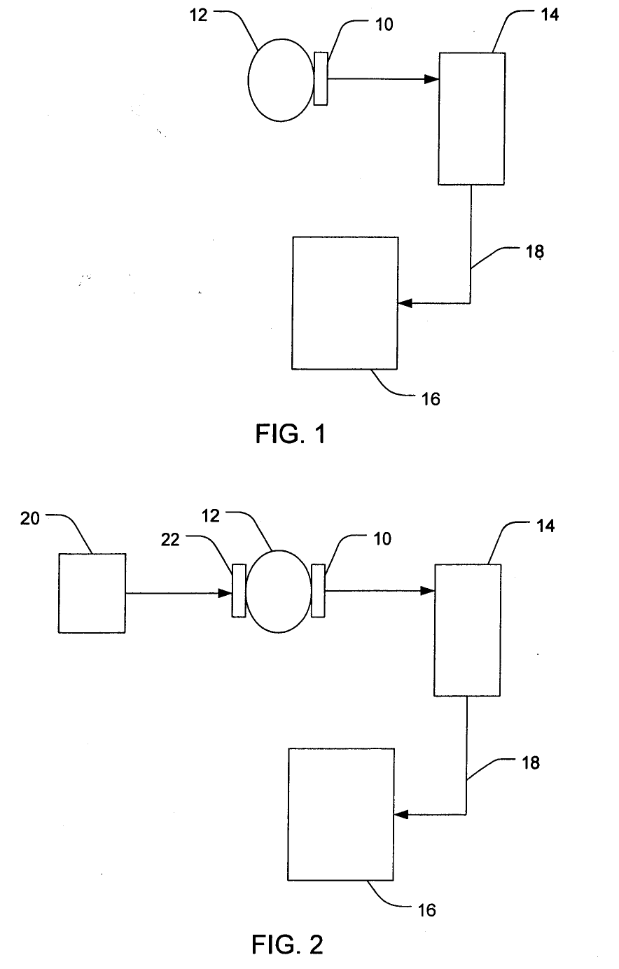

In one aspect, for example, the neurologic evaluation system may comprise a diagnostic system. Such a system may be utilized to identify a subject's neurologic states that are of an unknown origin. The system may also be used to assess static characterizations of neurologic states such as a stable state of depression. In some aspects, such a system may lack a stimulation element. One example of a diagnostic system is shown in FIG. 1. A neurologic monitoring interface 10 is functionally coupled to a subject 12 to gather multi-dimensional data related to at least one neurologic state. The neurologic monitoring interface 10 may vary depending on the form of monitoring being utilized and the neurologic state being evaluated. For example, electroencephalogram (EEG) monitoring may be accomplished with a neurologic monitoring interface 10 that includes surface electrodes, transdermal electrodes, or both. Physiological monitoring may utilize blood pressure cuffs, electrocardiogram (ECG) leads, etc. as the neurologic monitoring interface 10. Additionally, the neurologic monitoring interface 10 can be a single monitoring interface or multiple monitoring interfaces, depending on the type and/or number of monitoring devices being used.

The neurologic state monitoring element 14 receives input from the neurologic monitoring interface 10. Such a monitoring element may include active or passive sensors and elements coupled to a neural data processing interface 18. The neurologic state monitoring element 14 may include multiple different monitors to collect data concurrently from various different aspects of the neurologic system. As is discussed more fully below, the neurologic state monitoring element 14 may monitor a single or multiple neurologic states with a single or multiple monitoring methods or sensors. For example, the neurologic state monitoring element 14 may be configured to monitor both hypnotic depth and the analgesic state of the subject by utilizing one or more neurologic sensors. In another example, the analgesic state may be monitored by a single neural sensor such as an EEG, or by multiple neural sensors such as EEG and heart rate variability (HRV).

The neurologic state monitoring element 14 can be coupled to a neurologic data processing element 16 by the neural data processing interface 18. The neural data processing interface 18 receives, formats, and packages neurologic sensor data for transmission to the data processing element 16. Such an interface may be highly variable, depending on the combination of neurologic sensors and processing elements being utilized. Details regarding such an interface, however, are considered to be within the knowledge of one of ordinary skill in the art once in possession of the present disclosure. The neurologic data processing element 16 can process multi-dimensional data gathered from the neurologic sensors of the neurologic state monitoring element 14. Further details regarding the neurologic data processing element 16 are discussed below.

In another aspect, the neurologic evaluation system may comprise a basic therapy system. Such a system may provide non-automation supported application of appropriate stimulations based on a determination of actual neurologic states, and may be utilized in both clinical and non-clinical neurologic therapy settings. Examples of such neurologic therapy and treatment may include, without limitation, anesthesia, postoperative pain management, acute and chronic pain management, physical therapy, addiction treatment, etc. Additionally, such a system may be utilized for various psychotropic therapies and treatments including, but without limitation, sleep disorder therapy, depression therapy, anxiety therapy, etc.

One example of a basic therapy system is shown in FIG. 2. A typical basic therapy system may include a neurologic monitoring interface 10, a neurologic state monitoring element 14, and a neurologic data processing element 16 coupled to the neurologic state monitoring element 14 by a neural data processing interface 18, as described in FIG. 1. Additionally, the basic therapy system may include a neurologic stimulator 20 to produce neurologic stimulation in the subject 12. Such stimulation may be delivered to the subject 12 via a stimulation interface 22. The stimulation interface 22 may vary depending on the form of stimulation being utilized. For example, for pharmaceutical stimulation the interface may be an I.V. drip, an injectable or oral drug, a transdermal patch, etc. For non-pharmaceutical stimulation, the interface may be surface electrodes, implantable electrodes, a psychological test, etc.

In yet another aspect of the present invention, the neurologic evaluation system may comprise an automation supported therapy system. Such a system may provide automated therapy to a subject in a variety of clinical and non-clinical environments, with stimulation mechanisms being controlled fully or in part by feedback from data processing and control processing elements. Specific non-limiting neurologic therapies for which such automation may be beneficial include anesthesia, postoperative pain management, acute and chronic pain management, physical therapy, addiction treatment, etc. Additionally, such a system may be utilized for various phychotropic therapies and treatments including, but without limitation, sleep disorder therapy, depression therapy, anxiety therapy, immune system therapy, etc.

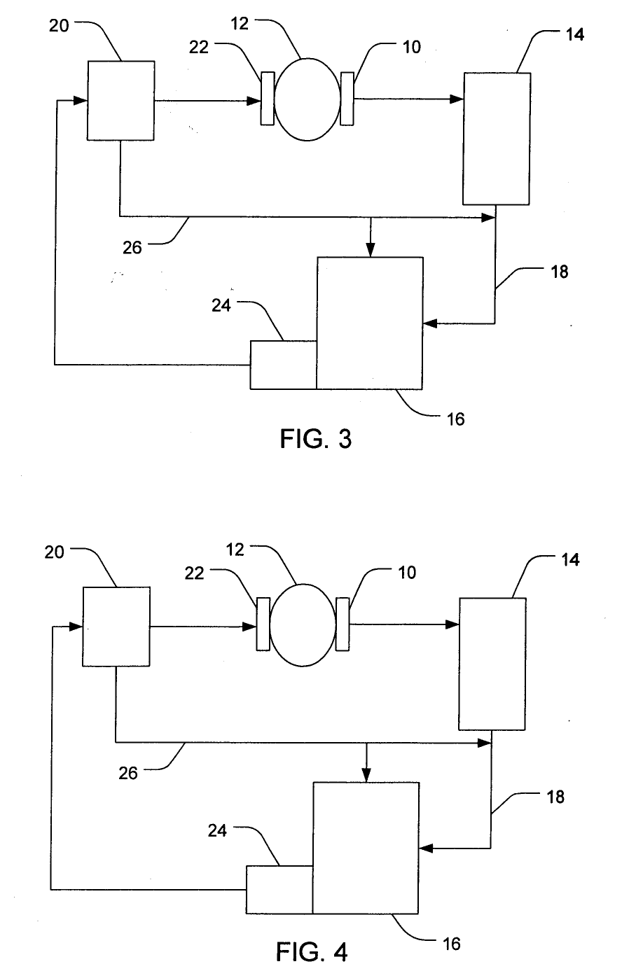

An example of an automated therapy system is shown in FIG. 3. Such a system may include a neurologic monitoring interface 10, a neurologic state monitoring element 14, a neurologic data processing element 16 coupled to the neurologic state monitoring element 14 by a neural data processing interface 18, and a neurologic stimulator 20 functionally coupled to the subject 12 via a stimulation interface 22 as shown in FIG. 2. The automated therapy system may also include a stimulation control processing element 24 to provide a feedback loop and thus allow modification of the stimulation provided to the subject 12 by the neurologic stimulator depending on the results of analyzed data. In one aspect, a data synchronization clock signal 26 may be functionally coupled to the neurologic stimulator 20, the neurologic data processing element 16, and the neurologic state monitoring element 14 in order to synchronize stimulation, monitoring and data analysis. Such a configuration may be utilized for clinical and non-clinical neurologic therapy and treatment including, but not limited to, anesthesia, postoperative pain management, acute and chronic pain management, physical therapy, addiction treatment, etc. In another aspect, such a configuration may be utilized for psychotropic therapy and treatment including, but not limited to, sleep disorder therapy, depression therapy, anxiety therapy, etc.

The above basic and automation supported therapy systems may include a neurologic response index as component of the neurologic data processing element 16 to facilitate the recognition and processing of specific neurologic states and support selection of appropriate therapeutic responses. The neurologic response index is a form of medical algorithm expert system using methods such as look-up tables, decision matrices, etc, to supplement and speed up data processing. It may be used to support the selection of appropriate evidence based medical therapies based on the available data representing specific neurologic states. In one aspect of the invention, the neurologic response index may be utilized to specify the type and form of neurologic stimulation for the stimulation control processing element 24. Medical informatics processes and methods such as the neurologic response index are known to those skilled in the art.

In a further aspect of the present invention, the neurologic evaluation system may comprise a neurologic research system. Such a system may be a stimulation-response research system employing multiple concurrent neuro-sensors and Relational Data Characterization (RDC) to characterize and depict specific neurologic responses to specific stimulation parameters. As is shown in FIG. 4, such a system may include a neurologic monitoring interface 10, a neurologic state monitoring element 14, a neurologic data processing element 16 coupled to the neurologic state monitoring element 14 by a neural data processing interface 18, and a neurologic stimulator 20 functionally coupled to the subject 12 via a stimulation interface 22 as shown in FIG. 3. The research system may also include a stimulation control processing element 24 to provide a feedback loop and thus allow modification of the stimulation provided to the subject 12 by the neurologic stimulator depending on the results of analyzed data, and a data synchronization clock signal 26 that may be functionally coupled to the neurologic stimulator 20, the neurologic data processing element 16, and the neurologic state monitoring element 14 in order to synchronize stimulation, monitoring and data analysis. Such a configuration may be utilized for various research tasks, including, but not limited to, investigation of stimulation parameter effects on neurologic states, advancing the discovery process in neuroscience, speeding up neuroscience research, optimizing stimulation parameters to achieve specific neurologic outcomes, etc.

Various neural states may be suitable for utilization in the various aspects of the present invention. It should be understood that no limitation is intended by the following discussion, and that any neural state is considered to be within the scope of the present invention. Examples of neural states that may be of interest may include, without limitation, hypnotic states including conscious hypnotic states and narcosis, analgesic states exemplifying various levels of pain perception, relaxation states, stress states, allostatic load, emotional states such as depression, happiness, sadness, fear, anxiety, etc., or combinations thereof. It is intended that the present invention encompass the monitoring of single and/or multiple monitoring states. As such, in one aspect, various combinations of neural states may be monitored simultaneously. In another aspect, a single neural state may be monitored with a single or multiple monitoring devices.

Various forms of stimulation are known to one of ordinary skill in the art, all of which would be considered to be stimulation within the scope of the present invention. Stimulation delivered by the neurologic stimulator may be pharmacological or it may be non-pharmacological. It is intended that the forms of stimulation described herein be merely exemplary and are not intended to be limiting.

For example, various forms of non-pharmacological stimulation are contemplated that may exert an effect on the neurologic system. Particular forms may have invasive, moderately invasive, and non-invasive applications. Other forms may be primarily invasive, primarily moderately invasive, or primarily non-invasive depending on the technology. For example, electrical stimulation is an example of a technology that can be practiced invasively, moderately invasively, or non-invasively. In invasive electrical stimulation, an area of neural tissue may be directly stimulated with electrical current. Moderately invasive stimulation may include epidermal or transdermal electrodes. Non-invasive electrical stimulation may include indirect electrical stimulation by means of surface electrodes and related technologies such as, without limitation, magnetic fields, electromagnetic radiation, capacitive coupling, etc.

In one aspect of the present invention, the neurologic system may be stimulated with a form of electrical current, either directly or indirectly. Depending on the complexity of the neurologic system, electrical current may allow the induction of various levels of anesthesia, analgesia, relaxation, etc. As has been discussed above, the electrical stimulation may be introduced to the neural system by invasive or non-invasive means. For example, the neural system can be electrically stimulated non-invasively via surface electrodes. Alternatively, the neural system can be electrically stimulated via invasive means. In such cases, the stimulation can be administered centrally or peripherally. One example of central neural stimulation may include deep brain stimulation, where an electrode is implanted directly in a subject's brain. An example of peripheral nerve stimulation may include vagus nerve stimulation, a technique whereby the subject's vagus nerve is stimulated peripherally.

Additionally, any form of electrical stimulation exhibiting a neurological effect on the neurologic system would be considered to be within the scope of the present invention. In one aspect, the electrical stimulation may include direct current. In another aspect, the electrical stimulation may include alternating current. In yet another aspect, the electrical stimulation may include both direct current and alternating current. Alternating currents can be single or multiple frequencies, and may include any type of waveform, including sinusoidals, partial sinusoidals, triangulars, ramp signals, square wave, gated pulse signals, asymmetrical, etc. In one aspect, gated pulse signals can have pulse widths of between about 0.5 seconds to about 10 nanoseconds, depending on the subject species and the particulars of the experiment being performed. The waveforms may also include unipolar or bipolar signals, with or without direct current offsets. Additionally, the waveforms may be voltage controlled or controlled current signals.

In another aspect of the present invention, stimulation of the neural system may be by pharmacological means. Various active agents are known to have stimulatory effects on many neurologic systems. Though much of the discussion herein is devoted to human pharmaceuticals and other techniques, it should be understood that the scope of the present invention includes non-human animals and insects, and that all pharmaceutically active agents may or may not be applicable, depending on the subject species. Accordingly, any pharmaceutically active agent that can exert a neural effect in any subject or species of subject is contemplated to be useful in the various aspects of the present invention. General examples may include, without limitation, analeptic agents, analgesic agents, anesthetic agents, anticholinergic agents, anticonvulsant agents, antidepressant agents, antihistamines, antihypertensive agents, antimigraine agents, antiparkinsonism agents, antipsychotic agents, antispasmodic agents, anxiolytic agents, attention deficit disorder and attention deficit hyperactivity disorder drugs, central nervous system agents, beta-blockers and antiarrhythmic agents, central nervous system stimulants, genetic materials, hypnotics, narcotic antagonists, nicotine, parasympatholytics, peptide drugs, psychostimulants, sedatives, steroids, sympathomimetics, tranquilizers, vasodilators, proteins, peptides, polypeptides, enzymes, and mixtures thereof. The active agents may be administered in any form known, such as, without limitation, oral forms, parenteral forms, transdermal forms, transmucosal forms, intravenous forms, intraarterial forms, aerosol forms, etc.

In yet another aspect of the present invention, the neurologic system may be stimulated with sensory stimulation. Sensory stimulation may be any form of stimulation that can exert an effect on the neurologic system of the subject, including, without limitation, aural, visual, somatosensory, psychological or emotional stimulation, etc., or combinations thereof. Certain types of stimulation may be categorized under multiple types of sensory stimulation. For example, various types of music can be classified as auditory stimulation as well as emotional or psychological stimulation.

A given form of stimulation may have a broad spectrum of effects in the biological system, or it may have more specific effects. For example, a drug given to a subject may exert effects throughout various neural regions and as well as regions of cardiac tissue. Another drug, however, may be very specific, predominantly affecting a single neural region. Similarly with electrical stimulation, large regions of neural tissue can be stimulated, or small localized or even single neurons may be stimulated without substantially effecting the surrounding neural environment.

Turning to neurological monitoring and neurologic state monitoring elements, the detection or measurement of neuro-states has previously been performed by very subjective means by monitoring gross changes in physiological measures such as physical movement, pulse rate, respiration, or subjective analogs like the Visual Analog Scale for pain measurement. To establish that a specific neurologic state results from given pharmacological or non-pharmacological stimulation variables, data would be collected from dozens to hundreds of controlled study trials to achieve acceptable levels of statistical significance. Various means to detect and quantify a specific neurologic state by more direct measurements of neurologic state changes can more rapidly indicate the high probability occurrence of a specific neurologic state and indicate a relative level of effect for all said states. Various aspects of the present invention allow the detection of changes in many neurologic states in a few seconds to a few minutes, whereas neurologic state determinations by conventional subjective research techniques are statistically determined over the course of several trials that may take days or weeks to complete and assess. Direct, objective, and concurrent neurologic state monitoring can provide a significant reduction in the time required to obtain useful data in certain types of neurologic state research.

As such, any means of detecting, sensing, monitoring, or measuring physiological or neurological responses that indicate and/or measure one or more neurologic states are considered to be within the scope of the present invention. In one aspect of the present invention, a single discrete neurologic state may be monitored with one or more neurologic monitoring means or devices. It may be particularly beneficial in the case of monitoring a single discrete neurologic state to perform such monitoring using at least two different monitoring methods. Two or more monitoring methods may be utilized to characterize different aspects of a specific neurologic state. For example, different monitors may be utilized in monitoring analgesia, one to characterize peripheral pain and another to characterize visceral pain. Additionally, the use of multiple monitors to monitor a single neurologic state may improve measurement accuracy. In another aspect, two or more discrete neurologic states may be detected and/or discriminated. Each neurologic state can be monitored via a single monitoring means or by multiple monitoring means as described above.

Accordingly, any means of functionally coupling or connecting neurologic sensors to the subject, to each other, or the interconnections between sensors and processing elements would be considered to be within the scope of the present invention. Such coupling may be by physical or non-physical means, such as, and without limitation, electrical wires, fiber optic cables, wireless communication, displacement current sensors, etc.

Various general categorizations of neurologic state monitors or sensors are contemplated. The following discussions of neurologic monitoring means is not intended to be limiting, but merely to provide examples of particular technologies that may be useful in practicing the various aspects of the present invention. As such, in one aspect, passive electrical neurologic state sensors may be utilized to employ passive electrical sensing to detect or measure neurological and/or physiological changes in a subject that can be used as an indicator of one or more discrete neurological states. Non-limiting examples may include electroencephalography (EEG), electromyography (EMG), electrocardiogram (ECG), etc. The processing of one or more neurologic sensor signals, or contiguous dependent data streams, such as ECG can produce additional indicators such as heart rate and blood pressure variability, pulse transit time, and vagal tone that reflect the state of the autonomic nervous system. Similar EEG signal processing approaches may be utilized to depict aspects of the central nervous system. For example, methods such as the Bispectral Index (BIS) of the subject's EEG may provide direct numerical indications of a subject's level of consciousness, or the hypnotic neuro-state, by analysis of EEG brain waves. Another method of monitoring the hypnotic state involves audio evoked potentials (AEP), where processed signals emitted from the brain stem are associated with audible stimuli. In another example, EMG devices may be utilized to assess states of stress and relaxation by analysis of motor unit potential, or α-motor neuron, responses. Additionally, in one aspect of the present invention, any passive method or sensor can be utilized that can respond with an indication of a change in a neurologic state within less than about 3 minutes of such a change. In another aspect, any passive method or sensor can be utilized that can respond with an indication of a change in a neurologic state in more than about 3 minutes after an occurrence of such a change. In yet another aspect, any passive method or sensor can be utilized that can respond with an indication of a change in a neurologic state within less than about 1 minute of such a change. In a further aspect, any passive method or sensor can be utilized that can respond with an indication of a change in a neurologic state within less than about 30 seconds of such a change.

Active electrical neurologic state monitors or sensors may also be utilized. In one aspect, active electrical neurologic state sensors may be utilized to employ active electrical sensing to detect or measure neurological and/or physiological changes in a subject that can be used as an indicator of one or more discrete neurologic states. Examples include, without limitation, bioimpedance measurements, galvanic skin response (GSR) impedance measurements, magnetic resonance imaging (MRI), positron emission tomography (PET) scans, etc. Such methods are known to those of ordinary skill in the art. Additionally, in one aspect of the present invention, any active method or sensor can be utilized that can respond with an indication of a change in a neurologic state within less than about 3 minutes of such a change. In another aspect, any active method or sensor can be utilized that can respond with an indication of a change in a neurologic state in more than about 3 minutes after an occurrence of such a change. In yet another aspect, any active method or sensor can be utilized that can respond with an indication of a change in a neurologic state within less than about 1 minute of such a change. In a further aspect, any active method or sensor can be utilized that can respond with an indication of a change in a neurologic state within less than about 30 seconds of such a change.

In another aspect of the present invention, evoked response neurologic state sensors may be utilized to employ evoked responses to detect or measure neurological and/or physiological changes in a subject that can be used as an indicator of one or more discrete neurologic states. Examples include, without limitation, audio evoked potential (AEP, used to characterize certain levels of consciousness), tail flick latency (TFL, used as a pain metric for rodent research), various forms of dolorimetry, and other evoked afferent response methods may be employed to assess, inter alia, the analgesic state of a subject. All of these methods of monitoring may be utilized as means to rapidly provide objective data about the status of various aspects of the nervous system and thereby can be utilized to indicate neurologic states. In one aspect of the present invention, any evoked response method or sensor can be utilized that can respond with an indication of a change in a neurologic state within less than about 3 minutes of such a change. In another aspect, any evoked response method or sensor can be utilized that can respond with an indication of a change in a neurologic state in more than about 3 minutes after an occurrence of such a change. In yet another aspect, any evoked response method or sensor can be utilized that can respond with an indication of a change in a neurologic state within less than about 1 minute of such a change. In a further aspect, any evoked response method or sensor can be utilized that can respond with an indication of a change in a neurologic state within less than about 30 seconds of such a change.

In yet another aspect of the present invention, physiological measurement neurologic state sensors may be utilized to detect or measure neurological and/or physiological changes in a subject that can be used as an indicator of one or more discrete neurologic states. Examples include, without limitation, blood pressure, pulse rate, respiration, etc. Such methods are known to those of ordinary skill in the art. Additionally, in one aspect of the present invention, any physiological measurement method or sensor can be utilized that can respond with an indication of a change in a neurologic state within less than about 3 minutes of such a change. In another aspect, any physiological measurement method or sensor can be utilized that can respond with an indication of a change in a neurologic state in more than about 3 minutes after an occurrence of such a change. In yet another aspect, any physiological measurement method or sensor can be utilized that can respond with an indication of a change in a neurologic state within less than about 1 minute of such a change. In a further aspect, any physiological measurement method or sensor can be utilized that can respond with an indication of a change in a neurologic state within less than about 30 seconds of such a change.

In a further aspect of the present invention, biochemical assay methods and associated sensors may be utilized to detect or measure neurological, physiological, or psychological changes in a subject that can be used as an indicator of one or more discrete neurologic states. Examples include, without limitation, blood chemistry analysis, neural tissue analysis, etc. Such methods are known to those of ordinary skill in the art. Additionally, in one aspect of the present invention, any biochemical assay method or sensor can be utilized that can respond with an indication of a change in a neurologic state within less than about 3 minutes of such a change. In another aspect, any biochemical assay method or sensor can be utilized that can respond with an indication of a change in a neurologic state in more than about 3 minutes after an occurrence of such a change. In yet another aspect, any biochemical assay method or sensor can be utilized that can respond with an indication of a change in a neurologic state within less than about 1 minute of such a change. In a further aspect, any biochemical assay method or sensor can be utilized that can respond with an indication of a change in a neurologic state within less than about 30 seconds of such a change.

In yet a further aspect of the present invention, interactive neurologic state assessments can be utilized to detect or measure neurological and/or physiological changes in a subject that can be used as an indicator of one or more discrete neurologic states. Any device or method employing interactive, written, verbal, or observational psychological assessments to detect or measure neurological state changes in a subject that can be used as an indicator of one or more discrete neurologic states. Examples include, without limitation, the Beck Depression Inventory (BDI) for depression, the State-Trait Anxiety Inventory for anxiety, the Agoraphobic Cognitions Questionnaire (ACQ) for fear, Visual Analogue Scales (VAS), etc. Such methods are known to those of ordinary skill in the art. Additionally, in one aspect of the present invention, any interactive neurologic state assessment method can be utilized that can respond with an indication of a change in a neurologic state within less than about 3 minutes of such a change. In another aspect, any interactive neurologic state assessment method can be utilized that can respond with an indication of a change in a neurologic state in more than about 3 minutes after an occurrence of such a change. In yet another aspect, any interactive neurologic state assessment method can be utilized that can respond with an indication of a change in a neurologic state within less than about 1 minute of such a change. In a further aspect, any interactive neurologic state assessment method can be utilized that can respond with an indication of a change in a neurologic state within less than about 30 seconds of such a change.

As has been discussed above, it is often difficult to envision effects produced by multiple interacting parameters. As a result of this, scientific research is often performed by linear investigation of only one parameter at a time—simply because it is the easiest approach and generally accepted as the conventional approach. The clinical assessment of medical and psychological conditions also tends to follow this linear paradigm. However, given the proper technology tools, it may be more effective to devise a set of experiments in which all pertinent input parameters are varied systematically and a range of results are collected in a few, rather than many, experiments. Such an experimental design may allow researchers to perform fewer experiments to obtain an adequate amount of data to answer research questions. Subsequent analysis of the resulting experimental data may identify optimal conditions, the parameters that most influence the results, the presence of interactions and synergisms, and so on. One potential problem with this method is that the results represent a complex multi-dimensional experimental data set that is difficult to envision. Fortunately, technologies are available that can keep track of thousands of parametric changes and interactions, and certain techniques have been developed to depict the relationships and interactions that occur during experiments in ways that allow researchers to see and better understand the processes involved. Such methods are commonly referred to as “data mining” techniques, and they are known to those skilled in the art.

The computational techniques and equipment used to process such multi-dimensional sets of possibly interacting data, and to depict the multi-dimensional relationships and interactions, may be referred to as relational data processing. It should be noted that no limitation is intended regarding the use of the terms “relational data processing” or “relational data characterization” (RDC). This terminology is intended to be construed broadly, to encompass any data processing technique that allows the analysis and depiction of relationships and interactions among multi-dimensional data to identify, discriminate, quantify, and otherwise characterize individual or multiple neurologic states.

The basic step in understanding data is to see relationships in that data. A scatter plot of two dimensional data plotted orthogonally displays the relationship between the two dimensions. A linear relationship between those dimensions results in a straight line. A circular relationship generates a circle. The names of classic geometries describe other familiar shapes. Such two dimensional visualizations in the graphical output data allow a researcher to envision relationships within the data that would be difficult to comprehend otherwise.

In one aspect of the present invention, techniques employing RDC concepts may be used to concurrently process and characterize multi-dimensional discrete or signal data. RDC finds and depicts the nature of relationships that exist within multi-dimensional data, and facilitates the ability to view functional relationships in multi-dimensional data sets. RDC may consist of any method of data processing that can concurrently processes multi-dimensional data sets to characterize one or many relationships that may occur within the multi-dimensional data sets as input variables change. This may be referred to as sensitivity analysis in data mining terminology. A variety of data mining applications have been developed that meet the requirements for RDC.

Additionally, in one aspect of the present invention, any RDC method can be utilized that can process neurologic sensor data to determine and indicate a change in a neurologic state within less than about 3 minutes of such a change. In another aspect, any RDC method can be utilized that can respond with an indication of a change in a neurologic state in more than about 3 minutes after an occurrence of such a change. In yet another aspect, any RDC method can be utilized that can respond with an indication of a change in a neurologic state within less than about 1 minute of such a change. In a further aspect, any RDC method can be utilized that can respond with an indication of a change in a neurologic state within less than about 30 seconds of such a change.

Furthermore, in one aspect of the present invention, any RDC method can be utilized that can that can identify or characterize a static or stable neurologic state within less than about 3 minutes of the functional connection to a subject via the appropriate neurologic sensors. In another aspect, any RDC method can be utilized that can identify or characterize a static or stable neurologic state in more than about 3 minutes after an occurrence of the functional connection to a subject via the appropriate neurologic sensors. In yet another aspect, any RDC method can be utilized that that can identify or characterize a static or stable neurologic state within less than about 1 minute of the functional connection to a subject via the appropriate neurologic sensors. In a further aspect, any RDC method can be utilized that can identify or characterize a static or stable neurologic state within less than about 30 seconds of the functional connection to a subject via the appropriate neurologic sensors.

Vector Fusion is one example of an RDC data processing method that facilitates the visualization and identification of data relationships that was developed by Robert Johnson, Ph.D. The composite relationships amongst the data are depicted in one complete image for all dimensions. Relationships existing in subsets of dimensions of data can also be discovered by vector-fusing subsets of dimensions. The functional relationships in the data are the relationships that exist relating each dimension one to another, regardless of whether or not those relationships were planned or programmed. Thus Vector Fusion captures the extrinsic properties of each dimension of data. As such, experiments with outcomes characterized by geometric or functional attributes are most likely to reveal curvilinear, geometric or line-locus (1:1) relationships in output data. One of ordinary skill in the art would have the ability to construct software capable of performing such data analysis once in possession of the present disclosure.

In various aspects of the present invention, data synchronization may prove helpful in subsequent data analysis. Providing a single synchronization clock signal which time stamps neurological stimulation, neurological state monitoring, and the values of data being collected may facilitate managing concurrent synchronized experimentation. Practical feasibility may be demonstrated by time-stamping the neuro-stimuli as they are applied in an experiment, and time-stamping each value of each dimension (or variable) being collected during the experiment.

Numerous hardware configurations are contemplated for accomplishing the RDC neurologic data processing described herein. Components such as central processors, firmware processors, data synchronization signal devices, visual displays, data storage, data transmission devices, user interfaces, calibration hardware, etc. would be readily understood by one of ordinary skill in the art once in possession of the present disclosure, and could thus be built with minimal experimentation.

### EXAMPLES

### Example 1

The following example is intended to be merely illustrative of the various aspects of the invention disclosed herein and is not intended in any way to limit the scope of the claimed invention. Other aspects of the invention that are considered equivalent by those skilled in the art are also within the scope of this invention.

Vector Fusion as an example describing time synchronization effects on discrete data for RDC: Vector Fusion maps a multidimensional vector y=f(x,w) where w is the two dimensional vector represented in formula 1:

w=w₁e^{iθ1}+w₂e^{iθ2}+ . . . w^{M}e^{iθM} Formula 1

and each wi is the value in each cell of M columns for each row of raw data. Each dimension (column) of raw data is assigned its own unique phase angle θt and the vector sum of all values wi is computed as the vector-fused resultant of all M component vectors. The vector sum is precise; there is no error in this mapping. The vector-sum here is the “approximating function” of statistical analyses. Other values of wi may duplicate this vector sum, but there is no error in the vector sum “approximating function” itself In vector-fusion, the approximating error ε is zero.

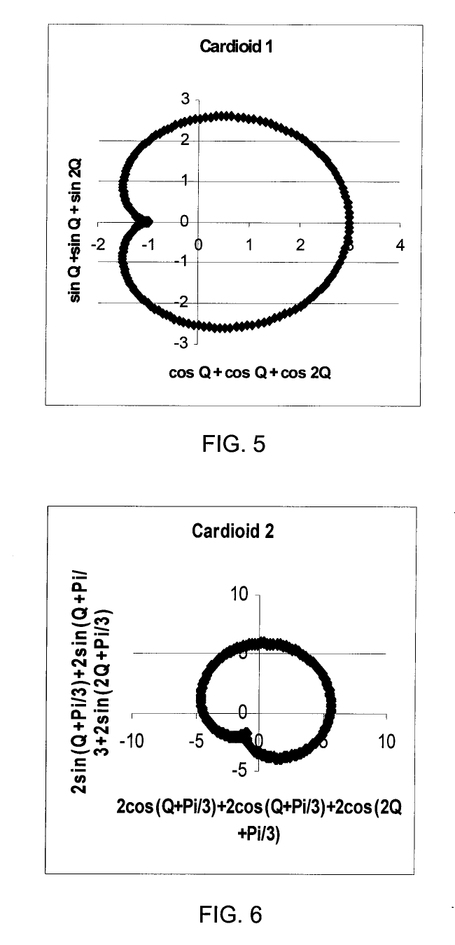

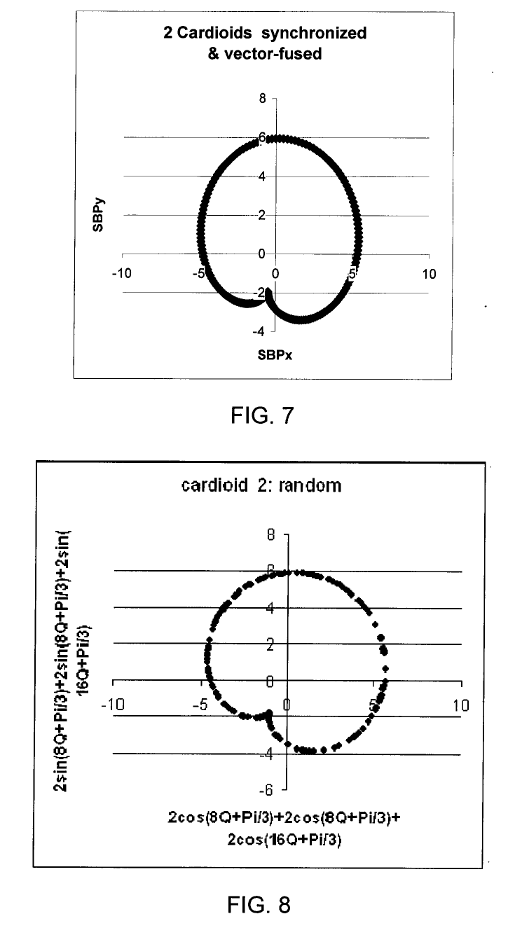

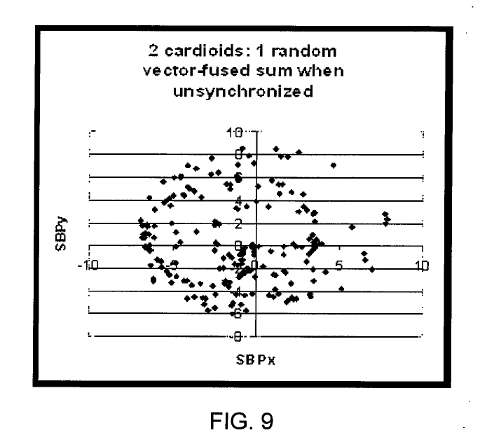

Significance of Time-synchronized data: Time-synchronized data is not often captured for data mining or learning applications. To understand the significance of synchronized data, FIGS. 5 and 6 show two cardioids of different diameters and orientations that are analyzed using vector-fusion with synchronized and then unsynchronized data. Four dimensions of data are generated using parametric equations when paired to describe the two cardioids of different diameters and rotated with respect to each other. FIG. 7 shows the vector-fused resultant cardioid generated from the two cardioids of FIGS. 5 and 6. To illustrate the effect of unsynchronized data, FIG. 8 is a cardioid generated by parametric equations similar to those of FIG. 6, but using randomly assigned values to Q. FIG. 9 shows the vector fused resultant “cardioid” generated from the two cardioids of FIGS. 5 and 8.

These results illustrate that synchronized data when analyzed with Vector Fusion displays the relationship between those four dimensions of data. Unsynchronized but otherwise similar data containing the same cardioid relationships reveals no obvious structural relationship in that unsynchronized data. The cluster of FIG. 6 is similar to clusters as found with statistical analysis. In statistics, the best analysis that can be performed is typically to find the center of such a cluster with regression analyses. With four dimensions of data, one would then do several regressions in order to separate the clusters generated with each regression. In such statistical analysis, it doesn't matter whether the data is synchronized or not because the analytical technique is unable to distinguish between the types of data.

With Vector Fusion and synchronized data, the precise relationship can be immediately apparent for synchronized cardioids, and thus researchers can predict or interpolate and thus visually understand the relationships with new or unknown points from new data. Without synchronized data, Vector Fusion, as one example of RDC, and statistical methods appear to be comparable.

### Example 2

In a study of short term analgesic pharmacodynamic responses, a swine of approximately 35-40 Kg was lightly sedated with isoflurane at MAC 1.0 for the following experiment. A low infusion of a paralytic agent (pavulon at 10 mg·hr was used to reduce occasional spontaneous movements that would affect data recordings. No other anesthetics were used, with the exception of boluses of remifentanil. Varied bolus doses of remifentanil were injected periodically into an IV line over 30 sec. to stimulate short term analgesic responses for data collection. Data collected included ECG, arterial BP (percutaneous), pulse oximetry, raw EEG, processed EEG, rectal temperature, CO2, etc. A minute by minute manual log of vitals data was kept to supplement instrumentation data recorded by two personal computer systems.

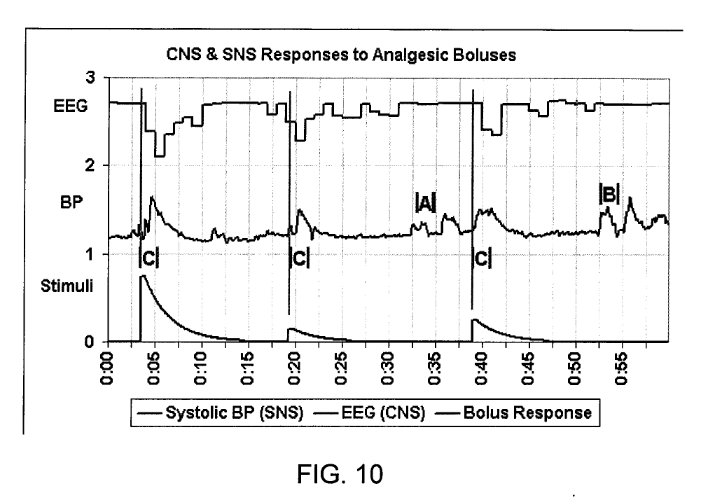

Referring to FIG. 10, a simple example of a RDC process is depicted by two separate neurologic responses to analgesic stimuli in the form of boluses of a fast acting opioid compound. The analgesic boluses typically cause a quick short term rise, bump, or hypertensive response in blood pressure (the C areas in FIG. 10) which may be interpreted as an analgesic response. This short term hemodynamic response reflects changes in the sympathetic nervous system associated with a rapid change in the analgesic state. This short term response effect may be counter-intuitive to some, since the general opinion is that remifentanil, albeit over longer time periods, produces a net hypotensive response. Such observations may lead a researcher to conclude that short term sympathetic blood pressure “bump” responses tend to be associated with analgesic bolus stimuli. However, occasional random short term elevations in blood pressure (A and B) also occur that are not associated with an analgesic bolus.

RDC analysis indicates that bumps in blood pressure are not the only responses that correlate with an analgesic bolus stimulus. Certain changes in the central nervous system (CNS) are associated with the analgesic response and this is reflected in processed EEG signal changes; these can be seen as inverted processed EEG bumps in the C areas of FIG. 10. However, the EEG data also is subject to occasional short term fluctuations that are not associated with a response analgesic stimulation. The RDC analysis results indicate that concurrent, and opposite, short term changes in both the EEG data AND blood pressure responses may provide a much more reliable indicator of a short term analgesic response than either the EEG or blood pressure responses alone. An ideal RDC system will adaptively discriminate dynamic, or “bump”, responses from slower shifts in static baseline data levels and process both the dynamic and static data.

As seen in the C areas in FIG. 10, both the blood pressure bumps and the EEG inverted bumps are consistently present and time synchronized relative to the analgesic bolus stimulation. In a Vector Fusion type of RDC processor, the vector sums for these dynamic analgesic stimulation responses will consistently fall within the same geometric area of a scatter plot, referred to as the “analgesic response zone”. In a situation where there is no analgesic stimulation and a random blood pressure bump occurs, a concurrent random EEG inverted bump event is unlikely to occur. In such cases the lack of a significant vector for an EEG response will skew the vector sum away from the analgesic response zone. Similarly, if there is an EEG response, but no blood pressure response, the lack of blood pressure response will also skew the vector sums outside of the analgesic response zone. Further RDC processing of the variables that contribute to vector sums mapping to, or away from, the analgesic response zone reveals that “concurrent response bumps” in both BP and EEG data tend to be associated with the analgesic boluses, but both responses do not tend to occur concurrently without an analgesic bolus, thus establishing, and characterizing, a strong relationship between the blood pressure AND EEG responses that are associated with the short term bolus infusion of an analgesic.

This simplistic example describes the basic role of RDC in aspects of the present invention, namely, to concurrently assess the multi-dimensional neurologic data that reflect various aspects of a complex neural system to find, and characterize, relationships within the data that correlate with input parameters and the resulting neurologic states. Using multi-dimensional data, i.e. multiple variables, rather than just one or two, can even more accurately identify, discriminate, and characterize neurologic events or states with RDC processing, thus associating specific nervous system activities with various types of responses in the data. As the specificity and diversity of neurologic sensors increase, the accuracy and reliability of RDC for mapping and characterization of neurologic states will also increase.

It should be understood that the above-described arrangements are only illustrative of the application of the principles of RDC as an element in the present invention. Numerous modifications and alternative arrangements may be devised by those skilled in the art without departing from the spirit and scope of the present invention. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method of assessing stimulatory effects on a neurologic system, comprising steps of:
stimulating the neurologic system;
monitoring at least one neurologic state for effects of said stimulation to the neurologic system;
gathering multi-dimensional data from the monitoring of the at least one neurological state; and
analyzing the multi-dimensional data to determine relationships between the stimulation and the effects on the at least one neurological state.

2. The method of claim 1, wherein the neurologic state is selected from the group consisting of hypnotic, analgesia, relaxation, stress, depression, anxiety, allostasis, immune responses, and combinations thereof.

3. The method of claim 2, wherein the neurologic state is analgesia.

4. The method of claim 1, wherein the neurologic state is multiple neurologic states.

5. The method of claim 1, wherein the step of analyzing the multi-dimensional data occurs in less than 3 minutes.

6. The method of claim 1, wherein the step of analyzing the multi-dimensional data occurs in less than 1 minute.

7. The method of claim 1, wherein the step of analyzing the multi-dimensional data occurs in less than 30 seconds.

8. The method of claim 1, wherein assessing stimulatory effects on a neurologic system occurs in a clinical setting.

9. The method of claim 1, wherein assessing stimulatory effects on a neurologic system occurs in a non-clinical setting.

10. The method of claim 1, wherein relationships between the stimulation and the effects include a correlation between the stimulation and at least two dimensions of data from the multi-dimensional data.

11. A method of assessing stimulatory effects on a neurologic system, comprising steps of:
stimulating the neurologic system;
monitoring with multiple monitors at least one neurologic state for effects of said stimulation to the neurologic system;
gathering data from the multiple monitors of the at least one neurological state; and
analyzing the data to determine changes in the neurological state due to the stimulation.

12. The method of claim 11, further comprising varying the stimulation of the neurologic system as a result of changes in the neurological state.

13. The method of claim 12, wherein the stimulation is varied automatically as a result of changes in the neurological state.

14. A system for assessing stimulatory effects on a neurologic system of a subject, comprising:
a neurological stimulator configured to be functionally coupled to the subject;
multiple neurological monitoring elements configured to be functionally coupled to the subject in order to monitor at least one neurologic state; and
a neurologic data processing element functionally coupled to the multiple neurological monitoring elements, said neurologic data processing element configured to analyze multi-dimensional data from the at least one neurologic state.

15. The system of claim 14, wherein the multiple neurological monitoring elements are configured to physically contact a skin surface of the subject.

16. The system of claim 14, wherein the multiple neurological monitoring elements are configured to not physically contact a skin surface of the subject.

17. The system of claim 14, wherein the neurologic data processing element is capable of analyzing the multi-dimensional data in less than 3 minutes.

18. The system of claim 14, wherein the neurologic data processing element is capable of analyzing the multi-dimensional data in less than 1 minute.

19. The system of claim 14, wherein the neurologic data processing element is capable of analyzing the multi-dimensional data in less than 30 seconds.

20. A method of monitoring a neurologic state of a neurologic system, comprising steps of:
monitoring at least one neurologic state of the neurologic system;
gathering multi-dimensional data from the monitoring of the at least one neurological state; and
analyzing the multi-dimensional data to evaluate the at least one neurological state.

21. The method of claim 20, wherein the neurologic state is selected from the group consisting of hypnotic, analgesia, relaxation, stress, depression, anxiety, allostasis, immune responses, and combinations thereof.

22. The method of claim 21, wherein the neurologic state is analgesia.

23. The method of claim 20, wherein of monitoring a neurologic state occurs in a clinical setting.
